(19) United States
(12) Patent Application Publication
(10) Pub. No.: US 2025/0142016 A1
(43) Pub. Date: May 1, 2025

(54) VIDEO OVERLAY SYSTEM FOR SURGICAL SETTINGS

(71) Applicant: Alcon Inc., Fribourg (CH)

(72) Inventors: Satish Kumar Calasapakkam GOPAL, Jurupa Valley, CA (US); Hideyuki YAGISAWA, Lake Forest, CA (US); Dan DURANT, Rancho Santa Margarita, CA (US)

(21) Appl. No.: 18/885,223

(22) Filed: Sep. 13, 2024

Related U.S. Application Data
(60) Provisional application No. 63/594,863, filed on Oct. 31, 2023.

(51) Int. Cl. H04N 5/272; A61B 90/00; H04N 7/10

(52) U.S. Cl. CPC H04N 5/272; A61B 90/361; H04N 7/10; A61B 2090/364; A61B 2090/372; A61B 2090/373

(57) ABSTRACT

A video overlay device for use in surgical settings to overlay surgical parameters from a surgical console over images from a surgical microscope is provided. The device includes a video input port configured to receive image data from a camera and a data port configured to receive a removable storage device storing encrypted WiFi configuration data. The device further includes a WiFi transceiver configured to connect, based on the encrypted WiFi configuration data, to a local WiFi network hosted by a surgical console and receive, from the surgical console, overlay data. The device further includes a processor, configured to generate, based on the image data and the overlay data, combined image data and a video output port configured to send the combined image data to a display.

### CROSS-REFERENCE TO RELATED APPLICATIONS

This application claim benefit of and priority to U.S. Provisional Patent Application No. 63/594,863, filed Oct. 31, 2023, which is hereby assigned to the assignee hereof and hereby expressly incorporated by reference in its entirety as if fully set forth below and for all applicable purposes.

### BACKGROUND

Typically, a surgical procedure, such as an ophthalmic surgical procedure, may be video recorded for documentation, research, learning, and/or teaching. For example, during a cataract surgery, a video camera and/or microscope may receive an image similar to the surgeon's view of the surgical area. Current techniques for cataract surgery include the use of ultrasonic energy in various modalities, vacuum at variable levels, irrigation of fluid and other variables that affect the surgical procedure and demonstrates different surgical techniques. These techniques and parameters are meaningful to the surgery and the surgeon may wish to provide these parameters in real time with the video signal from the video camera and/or microscope to a display to be recorded, used for training purposes, and/or used for documentation. Some data relating to parameters may remain constant during the surgical procedure, while others will change as the surgeon selects different settings during the surgical procedure.

Dedicated video overlay systems have been developed to perform the action of superimposing an image representing various parameters to the video signal captured from the surgical procedure. Operation of the video overlay systems often require a physical connection to the surgical console via a cable that transmits data to create a graphical representation of the data at the video overlay system. The video signal is similarly input into the video overlay system. The processed video output signal that is output from the video overlay system contains the video image as background with an overlying portion that display parameters from the surgical console.

Certain existing video overlay systems often require a physical connection via a data cable between the surgical console and the video overlay system to transmit data between the surgical console and the video overlay system. In certain other existing video overlay systems that do not require a physical connection between the video overlay system and the surgical console, there may be security concerns related to sharing WiFi (Wireless Fidelity) network and password information to configure the video overlay system to connect wirelessly to the surgical console.

### SUMMARY

Aspects of the present disclosure relate to a video overlay system for use in surgical settings, and more specifically, a video overlay device operable to overlay surgical parameters from a surgical console over images from a surgical microscope.

In certain embodiments, a device is provided. The device includes a video input port configured to receive image data from a camera and a data port configured to receive a removable storage device storing encrypted WiFi configuration data. The device further includes a WiFi transceiver configured to connect, based on the encrypted WiFi configuration data, to a local WiFi network hosted by a surgical console and receive, from the surgical console, overlay data. The device further includes a processor, configured to generate, based on the image data and the overlay data, combined image data and a video output port configured to send the combined image data to a display.

In certain embodiments, a method for generating video overlay data is provided. The method includes receiving, at a video input port, image data from a camera and reading, at a data port, encrypted WiFi configuration data stored on a removable storage device. The method further includes connecting, by a WiFi transceiver, to a local WiFi network hosted by a surgical console based on the encrypted WiFi configuration data and receiving, by the WiFi transceiver, overlay data from the surgical console. The method further comprises generating, at a processor, combined image data based on the image data and the overlay data and sending, by a video output port, the combined image data to a display.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates an example ophthalmic surgical system that may be used to perform ophthalmic procedures on an eye, according to embodiments of the present disclosure.

FIG. 2A illustrates a perspective view of a front side of an exemplary video overlay device, according to embodiments of the present disclosure.

FIG. 2B illustrates a perspective view of a back side of the exemplary video overlay device, according to embodiments of the present disclosure.

FIG. 3 illustrates a method for connecting the exemplary video overlay device to a surgical console, according to embodiments of the present disclosure.

FIGS. 4A to 4D illustrate graphical user interfaces (GUIs) associated with a method for pairing the exemplary video overlay device to a surgical console, according to embodiments of the present disclosure.

FIG. 5 illustrates a perspective view of an exemplary video overlay system, including the exemplary video overlay device, a surgical console, a microscope camera, and a television, according to embodiments of the present disclosure.

FIG. 6 illustrates combined image data presented on a display, according to embodiments of the present disclosure.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to a video overlay system for use in surgical settings, and more specifically, a video overlay system operable to overlay surgical parameters from a surgical console to a television projecting a video from a surgical microscope.

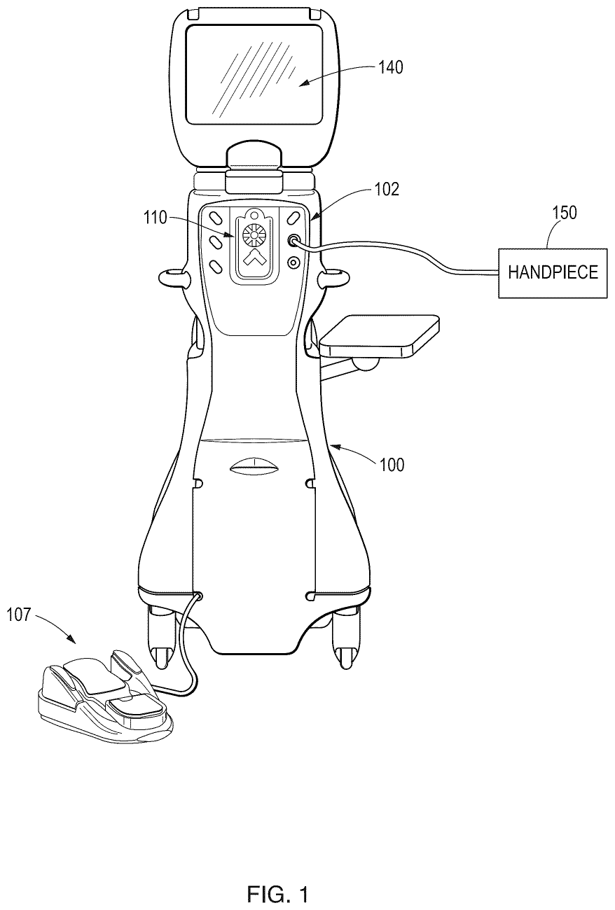

FIG. 1 illustrates ophthalmic surgical system 10 that may be used to perform ophthalmic surgical procedures on an eye, according to embodiments described herein. In the illustrated embodiments, ophthalmic surgical system 10 includes a surgical console 100 (or simply “console”), an interface device 107 (e.g., a foot pedal), and a hand piece 150. Surgical console 100 includes a housing 102, a display 140, and a fluidics subsystem 110.

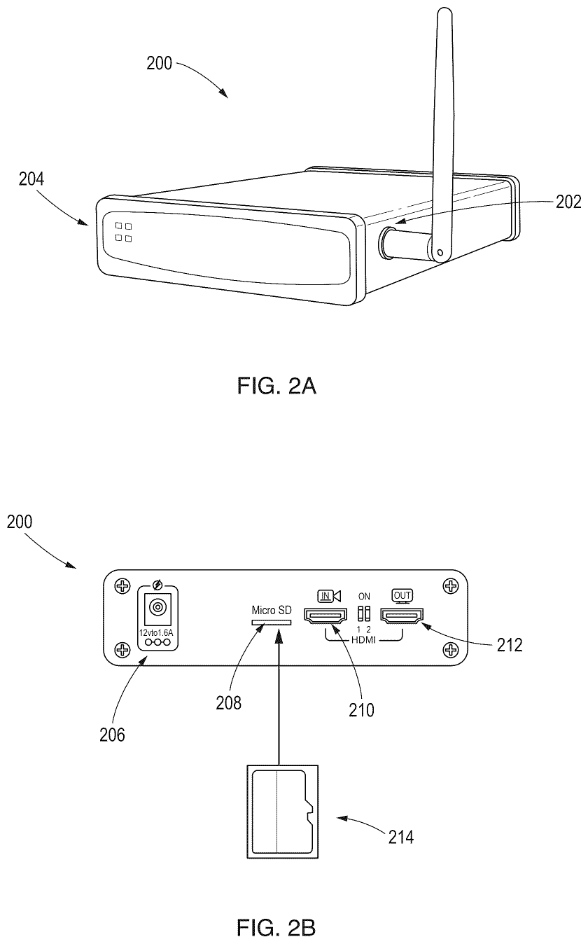

FIG. 2A illustrates a perspective view of a front side of a video overlay device 200. The front side view of the video overlay device 200 illustrates a WiFi transceiver 202, and a status indicator panel 204. The WiFi transceiver 202 may be configured to connect to a local WiFi network hosted by the surgical console (e.g., surgical console 100). Additionally, the WiFi transceiver 202 may be configured to receive data from the surgical console 100 over the WiFi network.

FIG. 2B illustrates a perspective view of a back side of a video overlay device 200. The back side of the video overlay device 200 illustrates a power input 206, a data port 208, a video input port 210, and a video output port 212. In certain embodiments, the data port 208 may be operable to receive a removable storage device 214. In certain embodiments, the removable storage device 214 may be a USB (Universal Serial Bus) drive or a microSD (micro Secure Digital) card. In certain embodiments, the removable storage device 214 may be preloaded in the data port 208 of the video overlay device 200 by the manufacturer. The preloaded removable storage device 214 may include the serial number of the video overlay device 200 preloaded on the removable storage device 214.

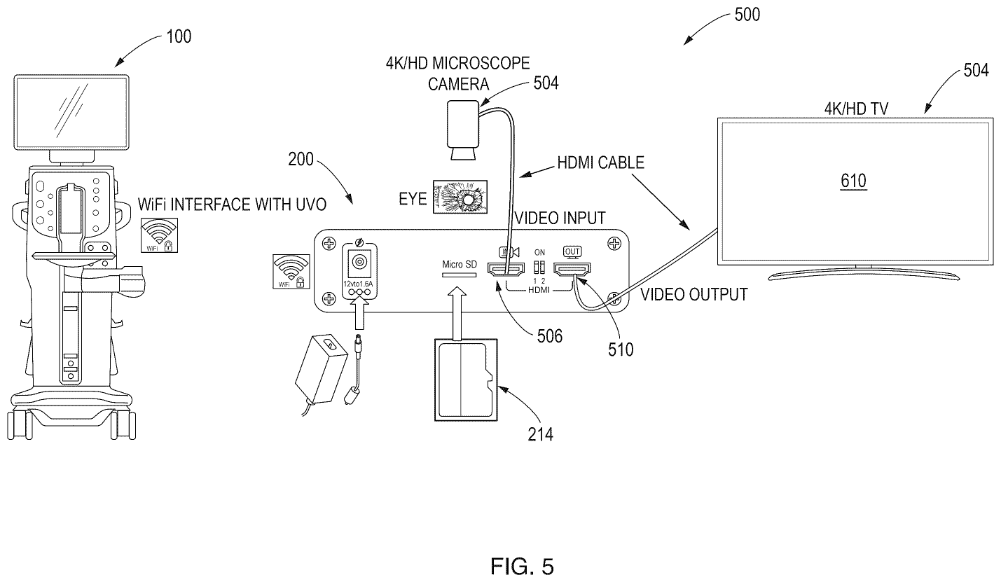

In certain embodiments, the video input port 210 and the video output port 212 may be High-Definition Multimedia Interface (HDMI) ports to couple, via wired connection, the video overlay device 200 to a microscope and a display device, as discussed herein in reference to FIG. 5.

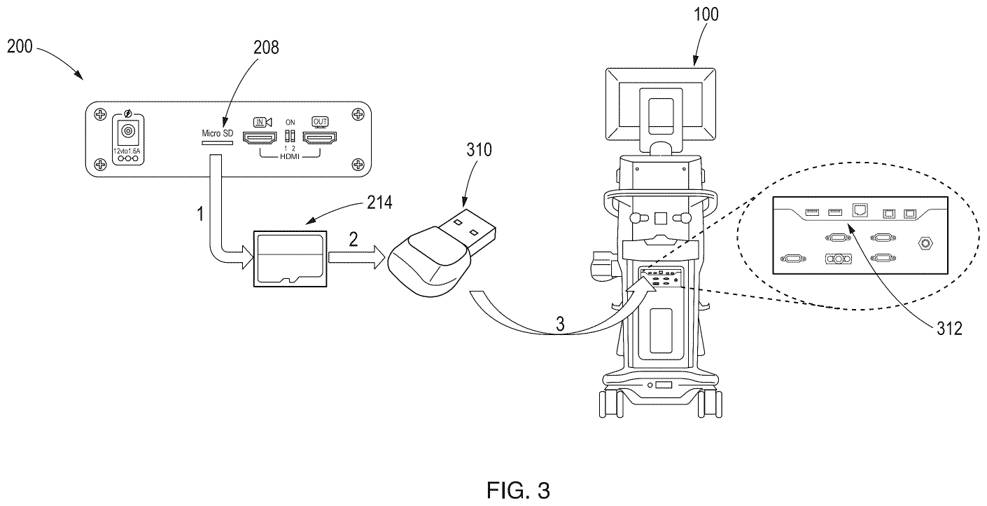

FIG. 3 illustrates a method for connecting a video overlay device 200 to a surgical console 100 via a local WiFi network, according to embodiments described herein. The method for connecting the video overlay device 200 to the surgical console 100 may begin by removing the removable storage device 214 (e.g., a microSD card) from a data port 208 of the video overlay device 200. As discussed in reference to FIG. 2B, the removable storage device 214 may be preloaded with the serial number of a video overlay device 200 to be provided to the surgical console 100.

The removable storage device 214 may be further configured to store encrypted WiFi configuration data when coupled the surgical console 100. In certain embodiments, encrypted WiFi configuration data may comprise WiFi network name, WiFi password, WiFi Region, WiFi frequency (e.g., 5 GHz (Gigahertz)/2.4 GHz), surgical console IP (Internet Protocol) address, WiFi security information, etc., of the local WiFi network hosted by the surgical console 100. By storing the encrypted WiFi configuration data on a removable storage device 214, the video overlay device 200 may connect to the WiFi network hosted by the surgical console 100 without requiring manual input of encrypted WiFi configuration data into the video overlay device 200. Further, the use of a removable storage device 214 may improve security of the WiFi network as the encrypted WiFi configuration data may not need to be provided to a user, and/or otherwise manually input into the video overlay device 200.

In certain embodiments, the removable storage device 214 may be a microSD card. The microSD card may be removed from the data port 208 and inserted into a USB microSD card reader 310 to couple the microSD card to the surgical console 100 via a USB input port 312 in the surgical console 100. In certain embodiments, the removable storage device 214 may be a USB, and the USB may be directly inserted into a USB input port 312 in the surgical console 100.

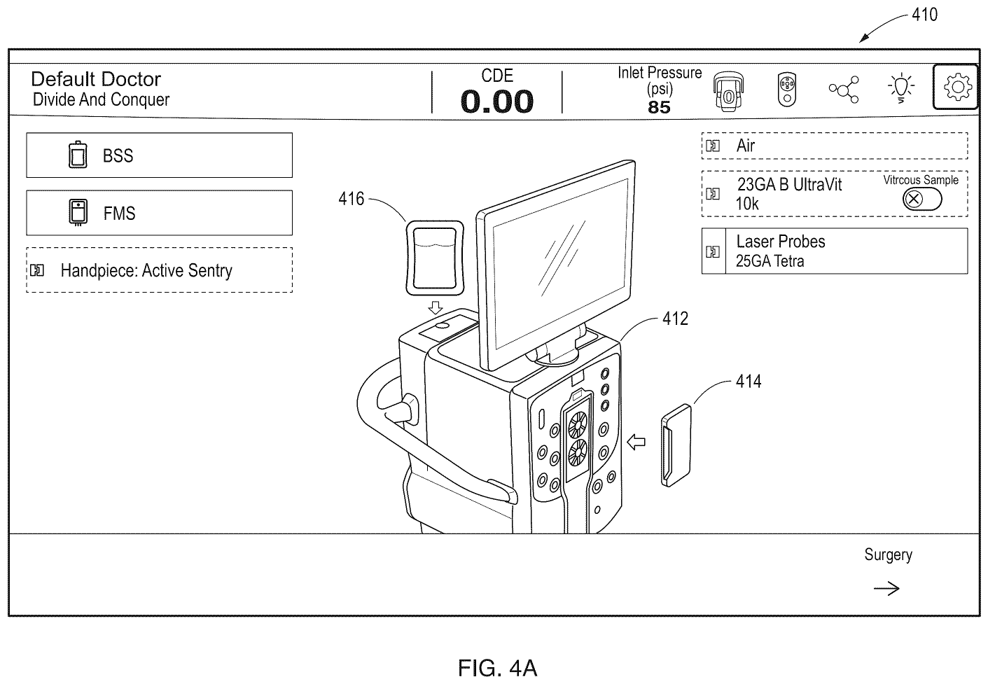

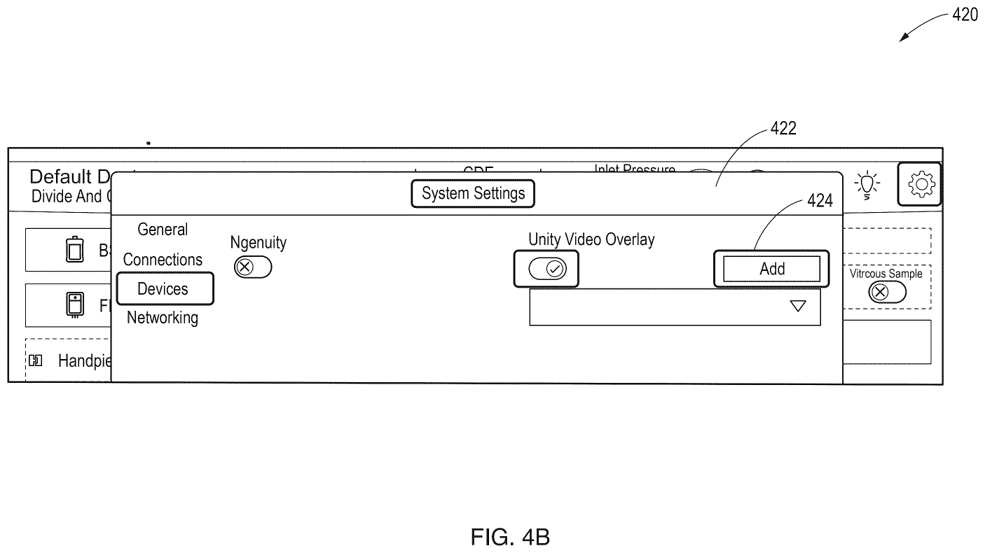

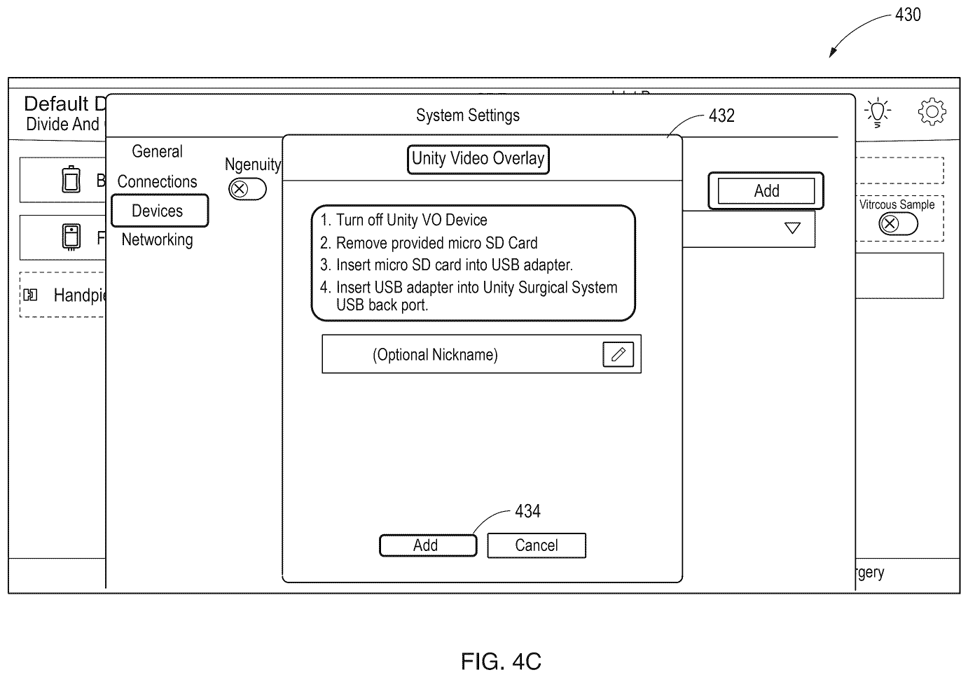

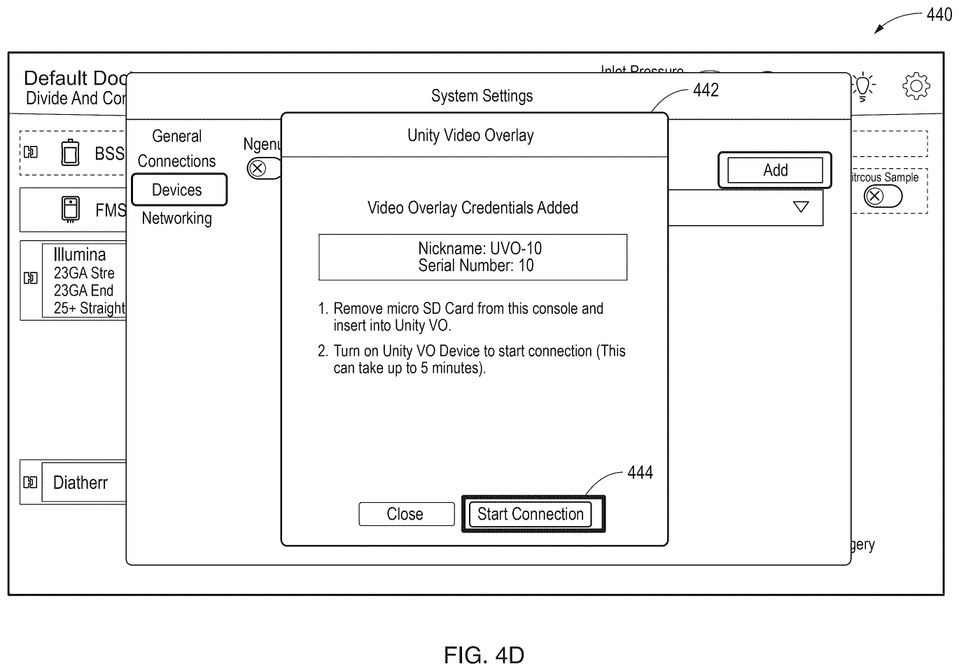

Upon insertion of the removable storage device 214 into the USB input port 312 of the surgical console 100, the surgical console 100 may guide a user through a series of steps to pair the video overlay device 200 to the surgical console 100 via the local WiFi network as described in reference to FIGS. 4A to 4D.

FIGS. 4A to 4D illustrate GUIs associated with a method for pairing the exemplary video overlay device to a surgical console, according to embodiments of the present disclosure.

A video overlay device 200 is paired, through a local WiFi network, to a surgical console 100, according to embodiments described herein. The method may begin through user input into a display of the surgical console (e.g., display 140), or upon insertion of a removable storage device into the USB port of the surgical console. The display 140 may guide the user to the video overlay connection page automatically upon insertion of the removable storage device 214.

FIG. 4A depicts GUI 410, which includes, inter alia, a graphical representation of a surgical console (e.g., surgical console 100), including housing 412, fluidics cassette 414, and fluid bag 416. GUI 410 may guide the user through various steps to prepare the surgical console for a specified surgical procedure (including specified surgical parameters), such as loading a fluid bag 416 and/or a fluidics cassette 414.

The user may select the settings wheel at the GUI 410 to reach GUI 420.

FIG. 4B depicts GUI 420, which includes, inter alia, pop-up window 422 and an “Add” button 424. The pop-up window 422 may be a “System Settings” window which populates in response to the user selecting the settings wheel of GUI 410.

The user may then select devices as presented in pop-up window 422 and locate the video overlay device 200 and click “Add.” The display 140 of the surgical console 100 may proceed to GUI 430.

FIG. 4C depicts GUI 430, which includes, inter alia, a pop-up window 432 and an “Add” button 434. The pop-up window 432 may be a “Video Overlay” window which populates with instructions for inserting the removable storage device 214 of the video overlay device 200 into a USB input port 312 of the surgical console 100.

GUI 430 may provide instructions to the user to “1. Turn off the video overlay device; 2. Remove the provided removable storage device from the video overlay device; 3. Insert the removable storage device into a USB adapter, if necessary; 4. Insert the USB adapter into the USB port of the surgical console.” Upon adding the removable storage device 214 from the video overlay device 200 to the surgical console 100 through the display 140, the surgical console 100 may proceed to GUI 440.

FIG. 4D depicts GUI 440, which includes, inter alia, a pop-up window 442 and a “Start Connection” button 444.

The method may begin at GUI 440 if the user inserts the removable storage device 214 from video overlay device 200 without navigating through GUIs 410, 420 and 430 as described herein. At GUI 440, the surgical console 100 may recognize the removable storage device 214 and record the serial number of the video overlay device 200 as stored on the removable storage device 214. The user confirms the credentials of the video overlay device 200 as shown on the pop-up window 442. The surgical console 100 may store encrypted WiFi configuration data on the inserted removable storage device 214. In addition to encrypted WiFi configuration data, the surgical console 100 may further store static image files (e.g., .PNG (Portable Network Graphic) files) and/or font files (.FNT files) on the inserted removable storage device 214. Certain image and font files (e.g., logos, panel backgrounds, indicator icons, etc.) may not be updated based on real time surgical parameters on the surgical console 100, which may allow those image and font files to be copied to the removable storage device 214 and provided to the video overlay device 200 via removable storage device 214 prior to a surgical procedure. However, in certain embodiments, image and font files may not be stored on the removable storage device 214 and instead transferred to the video overlay device 200 over the WiFi connection.

Once upload of the appropriate files and encrypted WiFi configuration data is completed through the display 140, the removable storage device 214 may be removed from the surgical console 100 and reinserted into the video overlay device 200. In certain embodiments, the user may follow the instructions provided to the user. For example, GUI 440 may instruct the user to remove the removable storage device from this console and reinsert into the video overlay device; and turn on the video overlay device to start the connection (This can take up to 5 minutes). Once the user has completed the instructions as presented on GUI 440, the user may select button 444 to initiate the connection between the video overlay device 200 and the surgical console 100.

The video overlay device 200 may access data stored on the removable storage device 214 (e.g., encrypted WiFi data and static image files and/or font files) when the removable storage device 214 is inserted into the video overlay device 200. Based on the encrypted WiFi configuration data present on the removable storage device 214, and the connection initiated through selecting button 444 presented on GUI 440, the video overlay device 200 may connect to the WiFi hosted by the surgical console 100.

Based on the static image files and font files, the video overlay device 200 may build a background overlay layer based on various images and fonts stored on the removable storage device 214. The background overlay layer may include fields, backgrounds, logos, indicator icons, etc. which may be configured to update according to real-time data from the surgical console 100. In certain other embodiments, the background overlay layer may be configured to receive a second overlay layer including real-time data from the surgical console 100. The multi-layer overlay configuration, including a background overlay layer and a second overlay layer, and the background overlay layer configured to update according to real-time surgical console data, are further described in reference to FIG. 5.

FIG. 5 illustrates a perspective view of a video overlay system 500, including a video overlay device 200 comprising a memory and a processor, a surgical console 100, a microscope camera 502, and a display 504, according to embodiments described herein. The video overlay system includes a video overlay device 200, coupled to a microscope camera 502 via a video input port 506 (e.g., an HDMI port) and a display 504 via a video output port 510 (e.g., an HDMI port). The video overlay device 200 may be further coupled to a surgical console 100 via a WiFi connection, as described herein.

In certain embodiments, the microscope camera 502 provides image data to the video overlay device 200 via a wired connection at the video input port 506. The image data may be a single image or a sequence of images (e.g., a video). The video overlay device 200 may accept image data and/or video data signals up to 3840×2160 resolution at a refresh rate of 60 times per second (e.g., 4K@60 Hz (approximately 4000 pixel horizontal resolution at 60 Hertz)). Further, the video overlay device 200 is configured to accept a wide variety of image and/or video data signals up to 4K@60 Hz (e.g., HD 1920×1080i (interlaced scan) 60 Hz to 1920×1080p (progressively displayed pixels) 25 Hz, etc.).

In certain embodiments, the surgical console 100 may communicate one or more commands to the video overlay device 200 via WiFi. The one or more commands may be text strings corresponding to a desired text or numerical output based on real-time surgical console data. For example, the real-time data may comprise at least one of a pulse value, a tors value, an infusion value, an aspiration value, a vacuum value, etc.

The one or more commands may further include a location and font for positioning the output. In certain embodiments, the one or more commands may be provided to the video overlay device 200 in alphanumeric characters, numbers, and letters.

Further, the surgical console 100 may communicate one or more resources to the video overlay device 200 via WiFi. In certain embodiments, the one or more resources may be parameter labels based on the categories of parameters in use by the surgical console. The video overlay device 200 may receive the one or more resources in a .PNG image format. The one or more resources may be provided to the video overlay device 200 in order to support various languages based on the system language on the surgical console 100. For example, the video overlay device 200 may support only ASCII (American Standard Code for Information Interchange) characters for display. By converting foreign language characters into a .PNG format, the foreign language characters may be displayed, even though the characters are not ASCII characters.

In certain embodiments, the one or more commands and/or one or more resources may be incorporated into the existing background layer (e.g., to create a single layer overlay data format), as discussed in reference to FIGS. 4A-4D. Alternatively, one or more commands and/or one or more parameters may be configured to create a second overlay layer to be overlaid to the existing background layer (e.g., to create a multi-layer overlay data format), as discussed in reference to FIGS. 4A-4D. In both examples, the commands and/or references may be periodically sent to the video overlay device 200 in order to accurately capture the real-time data as displayed on the surgical console 100, such as every 1 second, 2 seconds, 3 seconds, 5 seconds, 10 seconds, 15 seconds, etc. In other words, at some set frequency, the video overlay device 200 receives commands and/or resources which either requires editing the existing background overlay layer or updating a second overlay layer to overlay over the existing background overlay layer for display on display 504.

The multi-layer overlay data or the single layer overlay data may be received at the processor of the video overlay device 200. Additionally, the image data may further be received at the processor of the video overlay device 200. The processor may be configured to generate, based on the image data and the multi-layer or single layer overlay data, a combined image data 600.

The combined image data 600, including image data, as well as overlay data including static image files, font files, commands, and resources, may be sent to a display 504 through a video output port 510 within the video overlay device 200. In certain other embodiments, the combined image data 600 may be wirelessly sent to the display 504. In certain embodiments, the display 504 may be a widescreen monitor, an HDMI monitor, or a 4K monitor. The combined image data 600 sent to the display 504 may be recorded, used for training purposes, and/or used for documentation.

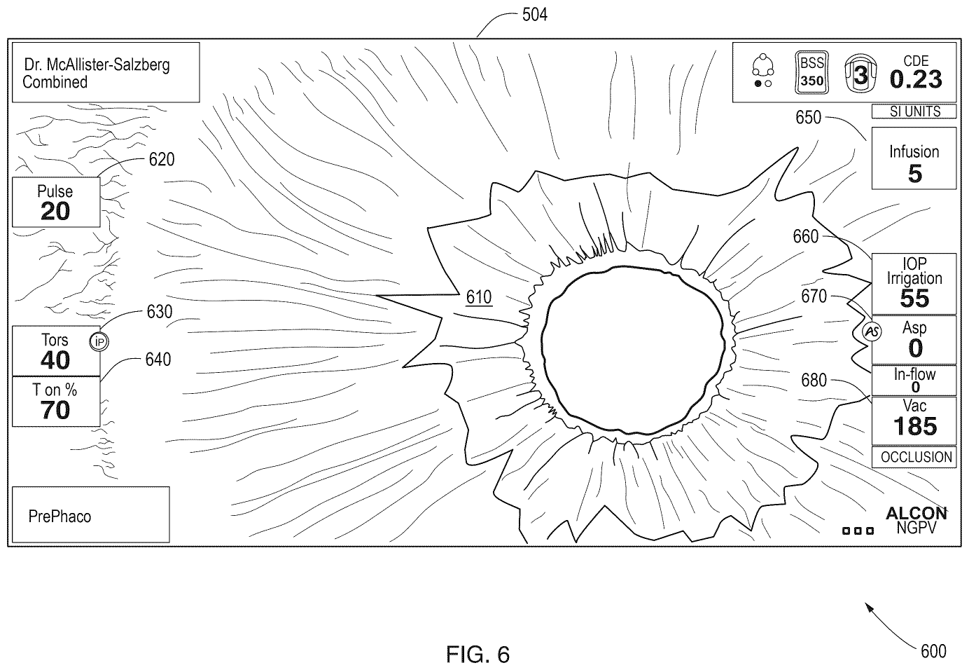

FIG. 6 illustrates combined image data 600 presented on display 504, according to embodiments of the present disclosure.

In certain embodiments, combined image data 600 may include image data 610, and overlay data such as Pulse 620, Tors 630, T on % 640, Infusion 650, IOP (Intraocular pressure) Irrigation 660, Aspiration 670, and Vacuum 680. The overlay data as described herein may relate to a phacoemulsification procedure. The overlay data may be different in various other procedures that may be performed using surgical console 100. In the phacoemulsification example, Pulse 620, Tors 630, and T_{ON}% 640 relate to the movement of the surgical instrument. Alternatively, Infusion 650, IOP Irrigation 660, Aspiration 670, and Vacuum 680 relate to the fluidics settings.

Beginning with the surgical instrument parameters, Pulse 620 represents the number of pulses per second of the surgical instrument (e.g., phacoemulsification probe). Pulse 620 may be a numerical value ranging from 5 to 200 pulses per second, for example. Tors 630 represents the torsional oscillation of the tip of the surgical instrument (e.g., phacoemulsification tip). Tors 630 may be a numerical value related to the frequency of the torsional oscillation of the tip of the surgical instrument. Tors 630 may be a numerical value ranging from 20 to 50 kilohertz (KHz), for example. T_{ON}% 640 may be a measure of torsional oscillation of the tip of the surgical instrument as a percentage of the maximum torsional oscillation. As such, T_{ON}% may be a numerical value from 1-100 representing the percentage of the maximum torsional oscillation the tip of the surgical instrument is currently oscillating at.

With regard to fluidics settings, Infusion 650 may be a measure of the flow rate of fluid from the fluid bag into the patient's eye during surgery. Infusion 650 flow rate may be a numerical value ranging from 0 to 50 cubic centimeters per minute (cc/min). IOP Irrigation 660 may be a measure of the intraocular pressure (IOP) within the patient's eye. IOP Irrigation 660 may be a numerical value ranging from 0 to 60 millimeters of mercury (mmHg), for example. Further, Aspiration 670 may be a measurement of fluid from the patient's eye during surgery. Aspiration 670 flow rate may be a numerical value ranging between 0 to 50 cc/min, for example. Vacuum 680 may be a measure of the vacuum pressure within the patient's eye. Vacuum 680 may be a numerical value ranging from 0 to 200 mmHg.

Embodiments of the present disclosure provide a video overlay system that includes a surgical console, a microscope camera, a video overlay device, and a display. The video overlay device is configured to receive image data from the microscope camera, receive overlay data through a wireless connection to a local WiFi network hosted by a surgical console, and output combined image data, including image data and overlay data, to the display.

In further embodiments of the video overlay system, the surgical console is connected to the video overlay device through a wireless connection.

In further embodiments of the video overlay system, the video overlay device comprises a data port operable to receive a removable storage device. The data port is a microSD port and the removable storage device is a microSD card. Alternatively, the data port is a Universal Serial Bus (USB) port and the removable storage device is a USB drive.

In further embodiments of the video overlay system, the removable storage device is inserted into the surgical console, and the surgical console directs a user through a series of prompts to store the surgical console encrypted WiFi configuration data on the removable storage device.

In further embodiments of the video overlay system, the overlay data comprises at least one of: static image files, font files, data related to real-time surgical console parameters.

The many features and advantages of disclosure are apparent from detailed specification, and, thus, it is intended by appended claims to cover all such features and advantages of disclosure which fall within scope of disclosure. Further, since numerous modifications and variations will readily occur to those skilled in art, it is not desired to limit disclosure to exact construction and operation illustrated and described, and, accordingly, all suitable modifications and equivalents may be resorted to that fall within scope of disclosure.

What is claimed is:

1. A device, comprising:
a video input port configured to receive image data from a camera;
a data port configured to receive a removable storage device storing encrypted WiFi configuration data;
a WiFi transceiver configured to:
connect, based on the encrypted WiFi configuration data, to a local WiFi network hosted by a surgical console; and
receive, from the surgical console, overlay data;
a processor configured to generate, based on the image data and the overlay data, combined image data; and
a video output port configured to send the combined image data to a display.

2. The device of claim 1, wherein:
the data port is a microSD port and the removable storage device is a microSD card; or
the data port is a Universal Serial Bus (USB) port and the removable storage device is a USB drive.

3. The device of claim 1, wherein the removable storage device is further configured to store at least one of static image files and font files.

4. The device of claim 1, wherein the overlay data comprises text strings corresponding to real-time data of the surgical console.

5. The device of claim 4, wherein the real-time data comprise at least one of: a pulse value, a tors value, an infusion value, an aspiration value, or a vacuum value.

6. The device of claim 1, wherein the encrypted WiFi configuration data may comprise a WiFi network name, a WiFi password, and WiFi security information.

7. The device of claim 1, wherein the image data received from the camera comprises a sequence of images.

8. The device of claim 1, wherein the image data received from the camera is a single image.

9. A method for generating video overlay data, comprising:
receiving, at a video input port, image data from a camera;
reading, at a data port, encrypted WiFi configuration data stored on a removable storage device;
connecting, by a WiFi transceiver, to a local WiFi network hosted by a surgical console based on the encrypted WiFi configuration data;
receiving, by the WiFi transceiver, overlay data from the surgical console;
generating, at a processor, combined image data based on the image data and the overlay data; and
sending, by a video output port, the combined image data to a display.

10. The method of claim 9, wherein:
the data port is a microSD port and the removable storage device is a microSD card; or
the data port is a Universal Serial Bus (USB) port and the removable storage device is a USB drive.

11. The method of claim 9, wherein the removable storage device is further configured to store at least one of static image files or font files.

12. The method of claim 9, wherein the overlay data comprises text strings corresponding to real-time data values.

13. The method of claim 12, wherein the real-time data values comprise at least one of: a pulse value, a tors value, an infusion value, an aspiration value, or a vacuum value.

14. The method of claim 9, wherein the encrypted WiFi configuration data may comprise a WiFi network name, a WiFi password, and WiFi security information.

15. The method of claim 9, wherein the image data received from the camera is a sequence of images.
